(19) United States
(12) Patent Application Publication
(10) Pub. No.: US 2004/0022735 A1
(43) Pub. Date: Feb. 5, 2004

(54) Enzyme activated contrast agents

(76) Inventors: Egidijus E. Uzgiris, Niskayuna, NY (US); Bruce Johnson, Scotia, NY (US)

Correspondence Address: Raymond E. Farrell, Esq., Carter, DeLuca, Farrell & Schmidt, LLP, Suite 225, 445 Broad Hollow Road, Melville, NY, 11747, US

(21) Appl. No.: 10/211,743

(22) Filed: Aug. 2, 2002

(51) Int. Cl. A61B 5/055

(52) U.S. Cl. 424/9.341; 435/40.5

(57) ABSTRACT

A contrast agent for magnetic resonance imaging is provided which is initially substantially neutral or negatively charged and which is acted upon by one or more enzymes to create a paramagnetic metal containing probe which is positively charged. The enzyme activated contrast agent localizes in areas containing the enzyme(s).

### BACKGROUND

1. Technical Field

This disclosure relates to diagnostic imaging techniques in which organs or a disease state in a subject may be imaged using a targeted contrast agent and to targeted contrast agents suitable for use in such techniques.

2. Description of Related Art

The use of non-invasive imaging techniques to visualize interior sections of living beings is a critical tool utilized by the medical profession in locating and diagnosing abnormalities and/or disease states. Magnetic resonance imaging (MRI) may be used for producing cross-sectional images of the body in a variety of scanning planes. MRI employs a magnetic field, radio frequency energy and magnetic field gradients to make images of the body. The contrast or signal intensity differences between tissues mainly reflect the T1 (longitudinal) and T2 (transverse) spin relaxation values and the proton density, which generally corresponds to the free water content, of the tissues. If MRI is performed without employing a contrast agent, differentiation of the tissue of interest from the surrounding tissues in the resulting image may be difficult. To change the signal intensity in a region of a patient by the use of a contrast medium, several possible approaches are available. For example, a contrast medium may be designed to change the T₁ and the T₂ relaxation time.

One of the mechanisms employed in MRI to provide contrast in reconstructed images is the T₁ relaxation time of the spins. After excitation, a period of time is required for the longitudinal magnetization to fully recover. This period, referred to as the T₁ relaxation time, varies in length depending on the particular spin species being imaged. Spin magnetizations with shorter T₁ relaxation times appear brighter in magnetic resonance (MR) images acquired using fast, T₁ weighted nuclear magnetic resonance (NMR) measurement cycles. A number of contrast agents which reduce the T₁ relaxation time of neighboring water protons are used as in vivo markers in MR images. The level of signal brightness, i.e., signal enhancement, in T₁ weighted images is proportional to the concentration of the agents in the tissue being observed.

Gd-DTPA (gadolinuim-diethylenetriaminepentaaceticacid) is an MRI contrast agent approved by the U.S. Food and Drug Administration which has been used to estimate angiogenic activity of tumors. However, this contrast agent is not ideal for characterizing tumor vasculature because it rapidly migrates to the extravascular space before being excreted through the kidneys. The tumor NMR signal measurements become delicate, being based on the dynamics of contrast agent uptake and elimination. Accordingly, staging of tumors by this approach may be difficult. The problem of rapid uptake and elimination is compounded by the fact that MR is relatively insensitive and near millimolar concentrations of paramagnetic ions are required.

To avoid the delicate dynamic aspects of Gd-DTPA uptake measurements, albumin—Gd-DTPA has been used as a macromolecular contrast agent in connection with vasculature studies of tumors. In this instance, the elimination process does not play a role in the observed MR signals, so that a much simpler and more reliable signal analysis is possible. There are however, several drawbacks to this approach. Permeability of tumor vasculature to albumin—Gd-DTPA is not high enough to produce large MR signal changes, thus limiting the sensitivity of this approach. The observable MR signal changes appear to be concentrated mainly at the rim of implanted tumors and a full volume assessment appears to be lacking. In addition, albumin—Gd-DTPA may cause associated immune reactions when injected.

An elegant approach for focal administration of Gd and/or drugs to tumors is disclosed in U.S. Pat. Nos. 5,762,909 and 6,235,264. A reptating polymer having a worm-like conformation, e.g., a homopolymer of lysine or polypeptides of poly-glutamic acid and poly-aspartic acid having a high number of the peptide residues, substituted with Gd-DTPA, is injected and remains in the vasculature as a blood pool agent. It leaks out of the endothelium only in tumors which have a hyperpermeable endothelium. The hyperpermeability is a result of angiogenesis signals emanating from tumor cells under nutrient and oxygen stress. The extravasation of the polymeric agents in the tumors is thought to be much higher than for the globular agents due to the process of reptation, which allows the polymers to migrate around obstacles in a small convective force field. The globular agents, on the other hand, cannot move through very small pores or around obstacles in a fibrous matrix of the basement membrane of the endothelium and are thus repelled and mostly remain in the blood circulation before being cleared out through the renal or hepatobiliary excretion channels. Hence, globular agents give small tumor signals and small signals of tumor permeability when injected intravenously.

International application WO 01/89584 is directed to contrast agent substrates which are said to change pharmacodynamic and/or pharmacokinetic properties upon a chemical modification from a contrast agent substrate to a contrast agent product in a specific enzymatic transformation, thereby detecting areas of disease upon a deviation in the enzyme activity from the normal. In one example, a microbubble preparation is described that is a substrate for a matrix metalloproteinase enzyme utilized to cleave an undeca-lipid-derivatized peptide containing an MMP-7 cleavage site. The enzyme cleaves the peptide in the vicinity of two neighboring leucines, liberating a peptide bearing a net negative charge of 2 and leaving a net positive charge which allows the microbubbles to bind to cell surfaces or extracellular matrix close to the site of charge alteration by the enzyme.

The quest for safe and efficient contrast agents useful in MRI is a continuing one. The ability to provide a high concentration of paramagnetic ions to desired locations in the body for suitable time periods is desirable.

### SUMMARY

A contrast agent is provided which includes a paramagnetic ion chelate having at least one substituent attached thereto, the substituent being a peptide having a site cleavable by an enzyme, wherein the overall charge prior to cleavage by the enzyme is substantially neutral or negative.

A substantially neutral or negatively charged contrast agent is provided which contains a portion containing a paramagnetic ion, the portion linked directly or indirectly to a negatively charged portion containing a peptide, wherein exposure to an enzyme cleaves the peptide to provide a moiety having a positive charge that includes the paramagnetic ion.

A method for imaging tissue in a subject is provided which includes administering to the subject a contrast agent which includes a paramagnetic ion chelate having at least one substituent attached thereto, the substituent being a peptide having a site cleavable by an enzyme, wherein the overall charge prior to cleavage by the enzyme is substantially neutral or negative; allowing sufficient time for the contrast agent to be exposed to the enzyme; and subjecting the tissue to magnetic resonance imaging.

A method for imaging tissue in a subject is provided which includes administering to the subject a substantially neutral or negatively charged contrast agent which contains a portion containing a paramagnetic ion, the portion linked directly or indirectly to a negatively charged portion containing a peptide, wherein exposure to an enzyme cleaves the peptide to provide a moiety having a positive charge that includes the paramagnetic ion; allowing sufficient time for the contrast agent to be exposed to the enzyme; and subjecting the tissue to magnetic resonance imaging.

A method for concentrating delivery of a contrast agent in tissue containing an enzyme of a subject is provided which includes administering to the subject a contrast agent containing a paramagnetic ion chelate having at least one substituent attached thereto, the substituent being a peptide having a site cleavable by the enzyme, wherein the overall charge prior to cleavage by the enzyme is substantially neutral or negative; and allowing sufficient time for the contrast agent to be exposed to the enzyme such that the peptide is cleaved by the enzyme to provide a positively charged contrast agent containing the paramagnetic ion, wherein the positively charged contrast agent is retained in the area of cleavage.

A method for concentrating delivery of a contrast agent in tissue containing an enzyme of a subject is provided which includes administering to the subject a substantially neutral or negatively charged contrast agent which contains a portion containing a paramagnetic ion, the portion linked directly or indirectly to a negatively charged portion containing a peptide, wherein exposure to the enzyme cleaves the peptide to provide a moiety having a positive charge that includes the paramagnetic ion; and allowing sufficient time for the contrast agent to be exposed to the enzyme such that the peptide is cleaved by the enzyme to provide a moiety having a positive charge that includes the paramagnetic ion, wherein the positively charged moiety is retained in the area of cleavage.

### BRIEF DESCRIPTION OF THE FIGURE

FIG. 1 is a schematic representation of a synthetic scheme for producing a contrast agent in accordance with the present disclosure.

### DESCRIPTION OF PREFERRED EMBODIMENTS

In accordance with the present disclosure, contrast agents are provided which are initially substantially neutral or negatively charged molecules. The contrast agents are administered to a subject and acted upon by enzymes present in certain tissues of the subject to provide a positively charged contrast agent which is retained in the tissue by virtue of electrostatic interaction between the positively charged agent and negatively charged tissue in the area of the enzyme. Accordingly, the contrast agent can be a relatively small molecule which readily diffuses in and out of all tissues where it is not sequestered by electrostatic attraction. In this manner, the contrast agent is concentrated in areas containing the enzymes and efficaciously cleared from areas that do not contain the enzymes. Where there is no attraction, the unsequestered contrast agent circulates and is flushed from the body. Focal accumulation of contrast agents in accordance with the present disclosure provides an expeditious modality for high resolution images since unsequestered contrast agent is rapidly cleared away from tissues without the enzymes. Tissues containing the enzymes are marked by the contrast agent and not obscured by leakage and/or retention of the contrast agent into surrounding tissue. Contrast agents herein are especially useful for magnetic resonance imaging of tumors since tumors contain enzymes not normally found in other tissues and connective tissue in tumors is negatively charged.

Contrast agents herein contain a paramagnetic ion chelate linked directly or indirectly to one or more substituents that, when acted upon by one or more enzymes, are cleaved and leave behind a positively charged molecule. Suitable paramagnetic ions include ions of transition and lanthanide metals (e.g., metals having atomic numbers of 6 to 9, 21-29, 42, 43, 44, or 57-71), in particular ions of Gd, Cr, V, Mn, Fe, Co, Ni, Cu, La, Ce, Pr, Nd, Pm, Sm, Eu, Gd, Tb, Dy, Ho, Er, Tm, Yb and Lu, preferably Mn, Cr, Fe, Gd and Dy, most preferably Gd.

As is well known, a chelating agent is a compound containing donor atoms that can combine by coordinate bonding with a metal atom to form a cyclic structure called a chelation complex or chelate. Conventional metal chelating groups may be used which are well known to those skilled in the art, e.g., linear, cyclic and branched polyamino-polycarboxylic acids and phosphorus oxyacid equivalents, and other sulphur and/or nitrogen ligands known in the art, e.g., DTPA, DTPA-BMA, EDTA (ethylenediaminetetraacetic acid), DO3A (1,4,7,10tetraazacyclododecane-N,N′,N″-triacetic acid), TMT, BAT and analogs, the N₂S₂ chelant ECD of Neurolite, MAG, HIDA, DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), THT 4′-(3-amino-4-methoxy-phenyl)-6,6″-bis(N′,N′-dicarboxymethyl-N-methylhydra zino)-2,2′:6′,2″-terpyridine), DOTA (1,4,7,10-tetraazacyclododecane-1,4,7,10-tetraacetic acid), OTTA (1-oxa-4,7,10-triazacyclododecane-N,N′,N″-triacetic acid); CDTPA (trans(1,2)-cyclohexanodiethylene-triamine-pentaacetic acid), etc. Particularly preferred chelating groups are DTPA and DOTA. Methods for metallating any chelating agents present are well-known. For example, metals can be incorporated into a chelant moiety by three general methods: direct incorporation, template synthesis and/or transmetallation.

Preferred chelates are susceptible to covalent bonding to one or more substituent side chains containing peptide bonds. In particular, peptide chains containing from about 1 amino acid to about 40 amino acids and which contain sequences which are cleaved by an enzyme that is overexpressed by a target tissue, such as, for example matrix metalloproteinases (MMPs). Such sequences are positioned within the peptide chain such that upon cleavage by the enzyme, the portion of the peptide remaining attached to the chelate imparts a positive charge to the chelate. Accordingly, it is preferred that the chain contains a proximate peptide portion ranging from about 1 to about 20 amino acids in length which is connected proximately to the chelate, a cleavage site, and a distal peptide portion attached after the cleavage site and which may preferably range from about 1 amino acids to about 20 amino acids in length.

In one embodiment, the proximate peptide portion consists of a peptide having its carboxy terminus connected to the chelate so that upon proteolysis the resulting terminus of the peptide chain attached to the chelate will be an amine which contributes a positive charge to the resulting chelate peptide species. The proximate portion may advantageously contain basic amino acids such as lysine and arginine that bear a positive charge under normal physiological conditions. Normal physiologic conditions are those conditions which typically occur in living beings. The distal peptide portion, which is removed upon cleavage with the enzyme, contains a balancing number of acidic amino acids such as aspartate or glutamate that bear a negative charge under normal physiologic conditions. By varying the ratio of acidic amino acids in the proximate portion to the number of basic amino acids in the distal portion, the overall charge can be regulated to impart a substantially neutral to negative charge to the contrast agent. In another embodiment, the proximate peptide portion consists of a peptide having its N terminus connected to the chelate.

It is preferred that cleavage sites recognized by MMPs are incorporated into peptide chain because MMPs are known to be over expressed by cancerous tumors. MMPs are a family of structurally related zinc-dependant endopeptidases collectively capable of degrading essentially all components of the extracellular matrix. There are at least 20 human MMPs known which have been divided into subgroups based on structure and substrate specificity, e.g., collagenases, stromelysins, matrilysins and gelatinases to name a few. Of those enzymes, MMP-2 and MMP-9, also called type IV collagenases or gelatinases, are related enzymes that break down gelatin, fibronectin and collagen. MMP-2 and MMP-9 cleave the sequence Pro-Leu-Gly≠Leu-X at the ≠ symbol wherein X is any amino acid, which is a preferred sequence for the cleavage site. Examples of suitable cleavage sites are Pro-Leu-Gly≠Leu-Phe, Pro-Leu-Gly≠Leu-Ala or Pro-Leu-Gly≠Leu-Trp. Suitable sequences also include Pro-Arg-(Ser/Thr)-(Leu/Ile)-(Ser/Thr), with cleavage taking place between the (Ser/Thr) and (Leu/Ile) residues. It should be understood that additional amino acids may be included on either side of the exemplified cleavage sites to extend the length of the cleavage site.

Peptide chains herein may be constructed by any means known to those skilled in the art. Automated solid phase synthesis is well known and particularly well suited to construct chains of suitable length. For example, standard Fmoc chemistry may be employed in a synthetic scheme.

The peptide chain may be attached to the chelate by any means known to those skilled in the art. For example, a mixed anhydride of the chelating group can be prepared according to the method as described in P. F. Sieving, A. D. Watson, and S. M. Rocklage, Bioconjugate Chem. 1. 65-71, (1990). The anhydride of a chelating group such as DTPA is then reacted overnight with a diamine (in which the diamine is in large excess to the anhydride). Ethylene diamine may be a suitable choice. The product is separated from the diamine and from DTPA which was not reacted, e.g., by ion exchange chromatography. The product has an amine group on one of the acetic acid arms of the pentaacetic acid structure of the DTPA. Linking this amine-DTPA product to the peptide chain may be accomplished by a carboxyl coupling method. The carboxy acid groups of the peptide can be activated by a coupling reagent, e.g., 1 ethyl-3-(3-dimethylaminopropyl) carbodiimide hydrochloride (EDC) (Pierce, Rockford, Ill.). It is preferred that only the end carboxy group of the peptide is activated. Fmoc solid state chemistry is a preferred method of accomplishing this. In addition, a peptide chain without carboxy side chains is suitable as well. Other coupling reagents well-known to those skilled in the art are commercially available from, e.g., Pierce. The activated group is then combined with the amine modified DTPA to produce an amide linkage of the DTPA to the peptide backbone as a sidechain. The end product may be separated by diafiltration. Additional substituent chains may be added to the chelating group by the same or other suitable methods known to those skilled in the art. In an alternative embodiment, the anhydride of the chelating group is reacted with the activated amino terminus of the peptide substituent to bond the amino terminus to the chelating group.

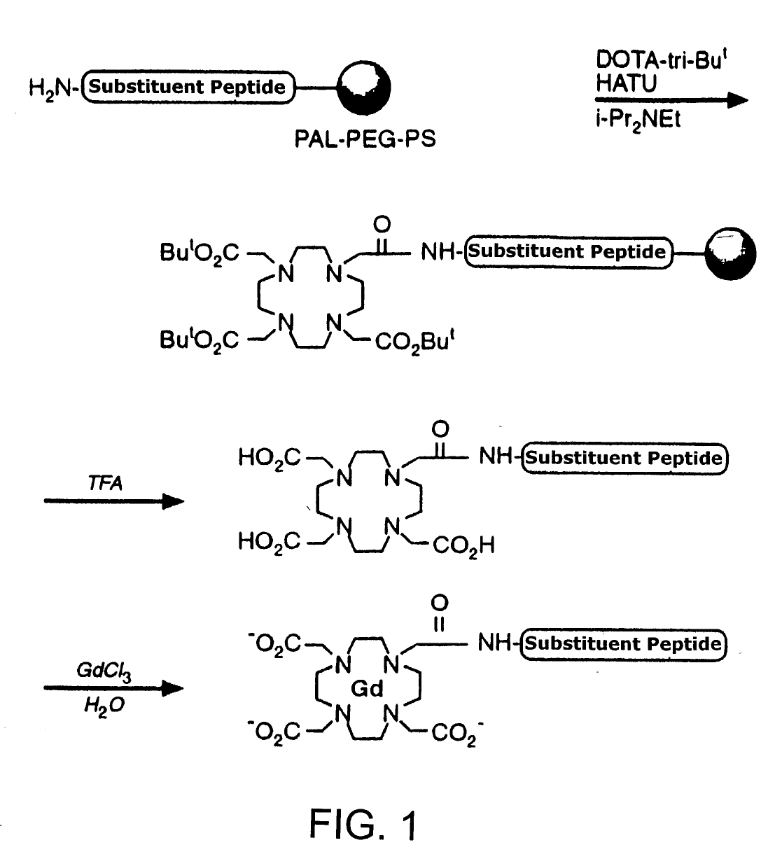

An example of a suitable Fmoc synthesis is illustrated in FIG. 1. A peptide substituent constructed by automated solid phase synthesis in accordance with the present disclosure is shown coupled to a polystyrene support. It is reacted with DOTA-tri-Bu^{t} and an activator such as O-(7-azabenzotrizol-1-yl)-1,1,3,3, tetra-methyluronium hexafluorophosphate (HATU) and i-Pr₂NEt to couple the peptide's amino terminus to the chelating group. The resulting product is removed from the polystyrene support using trifluoroacetic acid (TFA). The paramagnetic metal is added and chelated by the chelating group.

The contrast agents herein are initially advantageously substantially neutral or negatively charged to reduce agglutination and to allow for stable circulation in the blood prior to uptake in areas containing the enzymes. As used herein, “substantially neutral” is intended to mean that the net charge can be exactly neutral or nearly neutral. As used herein, “including”, “includes” and “include” are open ended terms and intended to mean including, but not limited to.

The contrast agents herein may be administered to patients for imaging in amounts sufficient to yield the desired contrast with the particular imaging technique. Generally, dosages of from 0.001 to 5.0 mmoles and preferably from 0.01 to 0.1 mmoles of chelated imaging metal ion per kilogram of patient bodyweight are effective to achieve adequate contrast enhancements. The contrast agents herein may be formulated with conventional pharmaceutical or veterinary aids, for example emulsifiers, fatty acid esters, gelling agents, stabilizers, antioxidants, osmolality adjusting agents, buffers, pH adjusting agents, etc., and may be in a form suitable for parenteral or enteral administration, for example injection or infusion or administration directly into a body cavity having an external escape duct, for example the gastrointestinal tract, the bladder or the uterus. Thus the contrast agents herein may be in conventional pharmaceutical administration forms such as tablets, capsules, powders, solutions, suspensions, dispersions, syrups, suppositories etc. Solutions, suspensions and dispersions in physiologically acceptable carrier media, for example, water for injection, will generally be preferred.

Contrast agents herein may therefore be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized.

For imaging of some portions of the body, the most preferred mode for administering contrast agents is parenteral, e.g., intravenous administration. Parenterally administrable forms, e.g. intravenous solutions, should be sterile and free from physiologically unacceptable agents, and should have low osmolality to minimize irritation or other adverse effects upon administration, and thus the contrast medium should preferably be isotonic or slightly hypertonic. Suitable vehicles include aqueous vehicles customarily used for administering parenteral solutions such as Sodium Chloride Injection, Ringer's Injection, Dextrose Injection, Dextrose and Sodium Chloride Injection, Lactated Ringer's Injection and other solutions such as are described in Remington's Pharmaceutical Sciences, 19th ed., Easton: Mack Publishing Co., pp. 1405-1412 and 1461-1487 (1995) and the United States Pharmacopeia-National Formulary (USP 25-NF 20) (2002). The solutions can contain preservatives, antimicrobial agents, buffers and antioxidants conventionally used for parenteral solutions, excipients and other additives which are compatible with the chelates and which will not interfere with the manufacture, storage or use of products.

In one embodiment, a subject is first imaged and then the contrast agent is introduced into the subject by injecting the contrast agent intravenously at approximately 0.025 mmoles Gd/Kg. The subject is then imaged, preferably beginning immediately after injection and at certain timed intervals. Preferably, the timed intervals are shortly after injection (within 10 minutes) and up to 1 hour post injection. An image at 24 hours may also be acquired.

The following examples are included for purposes of illustrating certain aspects of the subject matter disclosed herein and should not be interpreted as limiting the scope of the overall disclosure herein.

### EXAMPLE 1

DOTA mixed anhydride is prepared by adding 0.8 g of DOTA to 5 ml acetonitrilie and 1 ml of tetramethylguanidine and stirred until homogeneous. The solution is dried overnight over 4 angstrom molecular sieves. The solution is then decanted from the sieves and placed under a nitrogen atmosphere, cooled to −30° C. and stirred while adding 0.26 ml of isobutyl chloroformate (IBCF) slowly. The slurry is stirred for 1 hour.

Standard solid phase Fmoc chemistry is used (Rainin Symphony synthesizer) and protected amino acids from Advanced ChemTech, Louisville, Ky., to generate a peptide, Lys-Arg-Lys-Pro-Leu-Gly-Leu-Phe-Asp-Glu-Asp linked to resin and Fmoc protected at the amino terminus. The Fmoc is removed and the DOTA anhydride is added to the resin for coupling to the amino terminus. After 12 hours of stirring the peptide-DOTA products are acid cleaved from the resin and further purified.

Attachment to the carboxy end proximity is done by utilizing Lys(mtt) as the first amino acid on the resin of following sequence Glu-Gly-Gly-Pro-Leu-Gly-Leu-Phe-Lys-Gly-Lys(mtt). The amino group of Lys(mtt) is exposed with a weak acid while all the other amino acids remain protected, and the terminal amino is Fmoc protected. To this, the anhydride DOTA is added which links to the exposed amino group at the carboxy end of the peptide. This product is then cleaved and deprotected as above. Note the carboxy end is more direct way of leaving a positive charge behind—the NH2 group after cleavage remains with the chelator attached segment of the peptide.

### EXAMPLE 2

The penta anion of DTPA is prepared by reaction of DTPA (2.97 g, 7.56 mmol) with triethylamine (5.37 ml, 3.9 g, 38.56 mmol) in 35 ml acetonitrile for 50 min. and 55° C. under an inert atmosphere. Isobutylchloroformate (1.10 ml, 1.16 g, 8.47 mmol) is added dropwise to the DTPA penta anion, cooled in an well-equilibrated −45° C. bath, maintained by a Cryotrol temperature controller. After stirring at this temperature for 1 hour, the resulting thick slurry of the diethylenetriamine tetraaceticacid-isobutyl dianhydride is added dropwise, under ambient atmospheric conditions, to solid phase peptide constructs as described below.

Standard solid phase Fmoc chemistry is used (Rainin Symphony synthesizer) and protected amino acids from Advanced ChemTech, Louisville, Ky., to generate a peptide, Lys-Lys-Arg-Lys-Pro-Leu-Gly-Leu-Phe-Asp-Glu-Asp linked to resin and Fmoc protected at the amino terminus. The Fmoc is removed and the DTPA anhydride is added to the resin for coupling to the amino terminus. After 16 hours of stirring the peptide-DTPA products are acid cleaved from the resin and further purified.

The above description sets forth preferred embodiments and examples. It should be understood that those skilled in the art will envision modifications of the embodiments and examples that, although not specifically stated herein, are still within the spirit and scope of any claims which may be appended hereto.

What we claim is:

1. A contrast agent comprising a paramagnetic ion chelate having at least one substituent attached thereto, the substituent being a peptide having a site cleavable by an enzyme, wherein the overall charge prior to cleavage by the enzyme is substantially neutral or negative.

2. A contrast agent according to claim 1 wherein the paramagnetic ion is selected from the group consisting of Gd, Cr, V, Mn, Fe, Co, Ni, Cu, La, Ce, Pr, Nd, Pm, Sm, Eu, Gd, Tb, Dy, Ho, Er, Tm, Yb and Lu.

3. A contrast agent according to claim 1 wherein the paramagnetic ion is Gd.

4. A contrast agent according to claim 1 wherein the peptide comprises a proximate peptide portion having two ends, one end connected to the chelate, the other end connected to a cleavage site, the cleavage site having two ends, one end of the cleavage site connected to the proximate peptide portion, and the other end of the cleavage site connected to a distal peptide portion.

5. A contrast agent according to claim 4 wherein the proximate peptide portion comprises amino acids that are basic under physiologic conditions.

6. A contrast agent according to claim 4 wherein the distal peptide portion comprises amino acids that are acidic under physiologic conditions sufficient to balance positive charges from the chelate and proximate peptide portion and impart a substantially neutral or negative charge to the contrast agent.

7. A contrast agent according to claim 5 wherein the basic amino acids are selected from the group consisting of lysine and arginine.

8. A contrast agent according to claim 6 wherein the acidic amino acids are selected from the group consisting of aspartate and glutamate.

9. A contrast agent according to claim 1 wherein the paramagnetic ion chelate is made from a chelating group selected from the group consisting of DTPA, DTPA-BMA, EDTA (ethylenediaminetetraacetic acid), DO3A (1,4,7,10-tetraazacyclododecane-N,N′,N″-triacetic acid), TMT, BAT and analogs, the N₂S₂ chelant ECD of Neurolite, MAG, HIDA, DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), THT 4′-(3-amino-4-methoxy-phenyl)-6,6″-bis(N′,N′-dicarboxymethyl-N-methylhydra zino)-2,2′:6′,2″-terpyridine), DOTA (1,4,7,10-tetraazacyclododecane-1,4,7,10-tetraacetic acid), OTTA (1-oxa-4,7,10-triazacyclododecane-N,N′,N″-triacetic acid), and CDTPA (trans(1,2)-cyclohexanodiethylene-triamine-pentaacetic acid).

10. A contrast agent according to claim 1 wherein the paramagnetic ion chelate is made from a chelating group selected from the group consisting of DTPA and DOTA.

11. A contrast agent according to claim 2 wherein the cleavage site is a site which is cleaved by a matrix metalloproteinase.

12. A contrast agent according to claim 11 wherein the matrix metalloproteinase is MMP-2 or MMP-9.13. A contrast agent according to claim 4 wherein the amino terminus of the proximate peptide portion is connected to the chelate.

13. A contrast agent according to claim 4 wherein the carboxy terminus of the proximate peptide portion is connected to the chelate.

14. A method for imaging tissue in a subject comprising:
administering to the subject a contrast agent which includes a paramagnetic ion chelate having at least one substituent attached thereto, the substituent being a peptide having a site cleavable by an enzyme, wherein the overall charge prior to cleavage by the enzyme is substantially neutral or negative;
allowing sufficient time for the contrast agent to be exposed to the enzyme; and
subjecting the tissue to magnetic resonance imaging.

15. A method for imaging tissue in a subject according to claim 14 wherein the paramagnetic ion is selected from the group consisting of Gd, Cr, V, Mn, Fe, Co, Ni, Cu, La, Ce, Pr, Nd, Pm, Sm, Eu, Gd, Tb, Dy, Ho, Er, Tm, Yb and Lu.

16. A method for imaging tissue in a subject according to claim 14 wherein the paramagnetic ion is Gd.

17. A method for imaging tissue in a subject according to claim 14 wherein the peptide comprises a proximate peptide portion having two ends, one end connected to the chelate, the other end connected to a cleavage site, the cleavage site having two ends, one end of the cleavage site connected to the proximate peptide portion, and the other end of the cleavage site connected to a distal peptide portion.

18. A method for imaging tissue in a subject according to claim 17 wherein the proximate peptide portion comprises amino acids that are basic under physiologic conditions.

19. A method for imaging tissue in a subject according to claim 17 wherein the distal peptide portion comprises amino acids that are acidic under physiologic conditions sufficient to balance positive charges from the chelate and proximate peptide portion and impart a substantially neutral or negative charge to the contrast agent.

20. A method for imaging tissue in a subject according to claim 17 wherein the basic amino acids are selected from the group consisting of lysine and arginine.

21. A method for imaging tissue in a subject according to claim 17 wherein the acidic amino acids are selected from the group consisting of aspartate and glutamate.

22. A method for imaging tissue in a subject according to claim 14 wherein the paramagnetic ion chelate is made from a chelating group selected from the group consisting of DTPA, DTPA-BMA, EDTA (ethylenediaminetetraacetic acid), DO3A (1,4,7,10tetraazacyclododecane-N,N′, N″-triacetic acid), TMT, BAT and analogs, the N₂S₂ chelant ECD of Neurolite, MAG, HIDA, DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), THT 4′-(3-amino-4-methoxy-phenyl)6,6″-bis(N′,N′-dicarboxymethyl-N-methylhydra zino)-2,2′:6′,2″-terpyridine), DOTA (1,4,7,10-tetraazacyclododecane-1,4,7,10-tetraacetic acid), OTTA (1-oxa-4,7,10-triazacyclododecane-N,N′,N″-triacetic acid), and CDTPA (trans(1,2)-cyclohexanodiethylene-triamine-pentaacetic acid).

23. A method for imaging tissue in a subject according to claim 14 wherein the paramagnetic ion chelate is made from a chelating group selected from the group consisting of DTPA and DOTA.

24. A method for imaging tissue in a subject according to claim 14 wherein the cleavage site is a site which is cleaved by a matrix metalloproteinase.

25. A method for imaging tissue in a subject according to claim 24 wherein the matrix metalloproteinase is MMP-2 or MMP-9.

26. A method for imaging tissue in a subject according to claim 17 wherein the amino terminus of the proximate peptide portion is connected to the chelate.

27. A method for imaging tissue in a subject according to claim 17 wherein the carboxy terminus of the proximate peptide portion is connected to the chelate.

28. A method for concentrating delivery of a contrast agent in tissue containing an enzyme of a subject comprising:
administering to the subject a contrast agent containing a paramagnetic ion chelate having at least two substituents attached thereto, the first substituent being a positively charged moiety, the second substituent being a peptide having a site cleavable by the enzyme, wherein the overall charge prior to cleavage by the enzyme is substantially neutral or negative; and
allowing sufficient time for the contrast agent to be exposed to the enzyme such that the peptide is cleaved by the enzyme to provide a positively charged contrast agent containing the paramagnetic ion, wherein the positively charged contrast agent is retained in the area of cleavage.

29. A method for concentrating delivery of a contrast agent in tissue containing an enzyme of a subject according to claim 28 wherein the paramagnetic ion is selected from the group consisting of Gd, Cr, V, Mn, Fe, Co, Ni, Cu, La, Ce, Pr, Nd, Pm, Sm, Eu, Gd, Tb, Dy, Ho, Er, Tm, Yb and Lu.

30. A method for imaging tissue in a subject according to claim 28 wherein the paramagnetic ion is Gd.

31. A method for imaging tissue in a subject according to claim 28 wherein the peptide comprises a proximate peptide portion having two ends, one end connected to the chelate, the other end connected to a cleavage site, the cleavage site having two ends, one end of the cleavage site connected to the proximate peptide portion, and the other end of the cleavage site connected to a distal peptide portion.

32. A method for imaging tissue in a subject according to claim 31 wherein the proximate peptide portion comprises amino acids that are basic under physiologic conditions.

33. A method for imaging tissue in a subject according to claim 31 wherein the distal peptide portion comprises amino acids that are acidic under physiologic conditions sufficient to balance positive charges from the chelate and proximate peptide portion and impart a substantially neutral or negative charge to the contrast agent.

34. A method for imaging tissue in a subject according to claim 31 wherein the basic amino acids are selected from the group consisting of lysine and arginine.

35. A method for imaging tissue in a subject according to claim 31 wherein the acidic amino acids are selected from the group consisting of aspartate and glutamate.

36. A method for imaging tissue in a subject according to claim 28 wherein the paramagnetic ion chelate is made from a chelating group selected from the group consisting of DTPA, DTPA-BMA, EDTA (ethylenediaminetetraacetic acid), DO3A (1,4,7,10tetraazacyclododecane-N,N′,N″-triacetic acid), TMT, BAT and analogs, the N₂S₂ chelant ECD of Neurolite, MAG, HIDA, DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), THT 4′-(3-amino-4-methoxy-phenyl)-6,6″-bis(N′,N′-dicarboxymethyl-N-methylhydra zino)-2,2′:6′,2″-terpyridine), DOTA (1,4,7,10-tetraazacyclododecane-1,4,7,10-tetraacetic acid), OTTA (1-oxa-4,7,10-triazacyclododecane-N,N′,N″-triacetic acid), and CDTPA (trans(1,2)-cyclohexanodiethylene-triamine-pentaacetic acid).

37. A method for imaging tissue in a subject according to claim 28 wherein the paramagnetic ion chelate is made from a chelating group selected from the group consisting of DTPA and DOTA.

38. A method for imaging tissue in a subject according to claim 28 wherein the cleavage site is a site which is cleaved by a matrix metalloproteinase.

39. A method for imaging tissue in a subject according to claim 38 wherein the matrix metalloproteinase is MMP-2 or MMP-9.

40. A method for imaging tissue in a subject according to claim 31 wherein the amino terminus of the proximate peptide portion is connected to the chelate.

41. A method for imaging tissue in a subject according to claim 31 wherein the carboxy terminus of the proximate peptide portion is connected to the chelate.
